(19) United States
(12) Patent Application Publication
(10) Pub. No.: US 2005/0277859 A1
(43) Pub. Date: Dec. 15, 2005

(54) Joint protection device

(76) Inventor: Bruce S. Carlsmith, Sacramento, CA (US)

Correspondence Address: CASTLE & GROSS LLP, 11231 GOLD EXPRESS DRIVE, SUITE 100, GOLD RIVER, CA, 95670, US

(21) Appl. No.: 10/856,550

(22) Filed: May 27, 2004

(51) Int. Cl. A61F 5/00

(52) U.S. Cl. 602/20

(57) ABSTRACT

A device made in accordance with the present invention provides adequate joint protection for a user when the user is involved in activities wherein the joint is likely to have a limited impact area. This invention provides a joint protection device that is comfortable to wear because it is lightweight; well ventilated; designed to provide easily accommodate movement of the limbs without moving off the joint; and configured not to pinch or “bite” the user's skin. The present invention provides a joint protection device having a two-piece design, wherein an upper portion is pivotally connected to a lower portion with an opening between the portions. The device is removably attached to a user's joint with attachment members that are connected to the device and that wrap around the limb of the user. The device of the present invention does not substantially encompass the joint. Rather, the lower portion includes a substantially rigid portion that is operably configured to cover only a minimal area and preferably just the impact area of the joint, and the upper portion has a general u-shape that extends from opposing sides and above the joint.

### BACKGROUND OF THE INVENTION

1. Field of the Invention

The present invention relates to the field of joint protection devices, and more particularly to kneepads and the like.

2. Description of Related Art

There are many activities were the joints of a person, such as a knee or an elbow, come into contact with hard surfaces. Particularly, during many of these activities a person's joint impacts the surface, usually the ground, in a repetitive manner and often the joint remains in contact with the surface while supporting the weight of the person. These types of activities can be very uncomfortable to a person and may cause significant injury to the joints. There are many activities in the construction industry, for example, that require a person to operate on their knees, such as laying tile and carpet and the like.

There are a number of conventional devices that have been developed to protect joints. However, these conventional joint protection devices have numerous drawbacks and shortcomings. These devices are not comfortable to wear and tend to rotate or move in ways that are not desirable. Particularly, many of the prior art devices are not comfortable because they are: too heavy; not well ventilated; not designed to comfortably stay in place relative to the joint; and/or inappropriately designed in a way that the device actually pinches or “bites” the skin of the user.

### SUMMARY OF THE INVENTION

It is an object of the present invention to overcome the drawbacks and shortcomings of conventional joint protection devices. The present invention is a joint protection device that provides adequate joint protection for a user when the user is involved in activities where the joint is likely to have a limited impact area. These activities include for example, any activity where a user has to kneel. This invention provides a joint protection device that is comfortable to wear because it is, for example: lightweight; well ventilated; designed to provide an excellent fit on the joint; and configured not to pinch or “bite” the user's skin.

The present invention provides a joint protection device having a two-piece design, wherein an upper portion is pivotally connected to a lower portion with an opening between the portions. The device is removably attached to a user's joint with attachment members that are connected to the device and that wrap around the limb of the user.

Advantageously, the device of the present invention does not substantially encompass the joint. Rather, the lower portion includes a substantially rigid portion that is operably configured to cover only a minimal area and preferably just the impact area of the joint. This feature, as well as others, provides for adequate impact protection while only minimally increasing the weight of the overall device. As will be seen, the lightness of the device contributes to the device's ability to remain in position on the joint regardless of the motions of the user.

The upper and lower portions of the device of the present invention are configured such that they are substantially non-overlapping. Particularly the portions do not overlap over the front of the joint. Rather, the device is configured such that the opening of the device is positionable so as to expose the front of the joint when the device is worn; one can strap it on and it finds its own unique and proper position. Moreover, various embodiments of the present device include ventilation holes formed in the upper and lower portions. These features, as well as others, advantageously increase the ventilation of the device of the present invention. This ventilation helps reduce the amount of perspiration that the user experiences and with the reduced perspiration comes more comfort and a better continuous fit of the device.

There are many features of the present invention that enable a better fitting device than the prior art. For example, the pivot connections of the present device are preferably disposable at substantially opposing sides of the joint and substantially coaxial with the pivot axis of the joint. This feature, among others, allows the two parts of the device to move freely with and stay closely coupled with the two limbs that connect at the joint. The result is a device that does not constrict movement or chafe.

Additionally, this pivot feature advantageously helps to prevent the device from twisting around the limb of the user or sliding down off the joint. The upper portion is operably configured by its shape and attachment to the lower portion, as well as the limb, to slightly pull the lower member up toward the joint whenever the limb is flexed. This keeps the device in a proper position on the joint during use.

Another example is the position of the attachment member. In an embodiment wherein the device is a knee protection device, the attachment member that attaches the lower portion to the limb is preferably configured to be positioned above the calf muscle, thereby preventing the device from slipping down the leg.

Finally, a third feature provides a means for the upper member to be retained above the joint even when the joint is straight. The upper portion is operably configured to have a lower edge that rests against a top portion of the joint, such as the knee cap, so as to prevent the device from slipping.

The present invention, in a preferred embodiment, includes padding between areas of the portions of the device and the skin so as to prevent the pinching of the skin. In a preferred embodiment, the lower portion includes a rigid impact member and a pad. The impact member is configured to cover a limited impact area of the joint and the pad is larger so as to protect more of the limb from being scraped and the like by objects on the ground.

In accordance with a preferred embodiment, the present invention provides a protection device for a joint of a limb comprising: an impact member being substantially rigid and operably configured to cover a lower portion of the joint when the device is worn; a support band pivotally attached to the lower member by at least two pivot connections, wherein the support band is operably configured to cover a portion of the upper section of the limb above the joint defining an opening in the device between the impact member and the support band such that a front portion of the joint is exposed when the device is worn; and first and second retaining members, at least one of the first and second members removably connected to at least one of the impact member and support band and the other of the retaining members removably connected to at least the other of the impact member and support band, the first and second retaining members being operably configured to retain the impact member and support band adjacent the joint when the device is worn.

In another preferred embodiment, the present invention provides a joint protection device for a joint of a limb, comprising: a lower member being substantially rigid and having two pivot connections, wherein when the device is worn the lower member is operably configured to cover a portion of the joint and the two pivot connections are positioned adjacent opposing sides of the joint; an upper member having a length defined by opposing ends, the upper member has a generally inverted u-shape disposable about a portion of the joint, and one of the opposing ends is pivotally connected to one of the pivot connections and the other opposing end is pivotally connected to the other pivot connection, wherein when the device is worn the upper member extends from one of the pivot connections above the joint to the other of said pivot connections; and first and second retaining members, the first retaining member being releasably attached to the lower member and the second retaining member being releasably attached to the upper member, wherein when the device is worn the retaining members are operably configured to retain the lower and upper members adjacent to the limb.

In yet a further preferred embodiment, the invention provides a protection device for protecting a joint, which provides a pivotal connection between upper and lower portions of a limb, the device comprising: a lower member having a substantially rigid shell and a pad, the shell having a dimensional area that is less than a dimensional area of the pad, the pad having interior and exterior surfaces, the interior surface operably configured to be disposed adjacent the joint and the lower portion of the limb when the device is in use, and the shell disposed on the exterior surface; an upper member rotatably connected to the lower member, wherein the upper member does not cover any portion of the exterior surface of the pad, and when the device is worn the upper member is operably configured to cover a portion of the upper portion of the limb; and, retaining straps releasably connected to at least one of the lower and upper member and being operably configured to hold the device against the limb when the device is worn.

In yet another embodiment, the invention provides a pivotal connection between upper and lower portions of a limb. The device comprises an impact member being substantially rigid and operably configured to cover a lower portion of the joint when the device is worn. The device also comprises an upper support band pivotally attached to the impact member by at least two pivot connections, wherein the support band is operably configured to cover a portion of the upper section of the limb above the joint defining an opening in the device between the impact member and the support band such that a front portion of the joint is exposed when the device is worn. A lower support band is releasably connected to the limb and fits between the limb and the impact member such that the impact member does not contact the limb.

These and other features and advantages of this invention are described in, or are apparent from, the following detailed description of various exemplary embodiments of the devices and methods according to this invention.

### BRIEF DESCRIPTION OF THE DRAWINGS

Various exemplary embodiments of this invention will be described in detail, with reference to the following figures, wherein:

FIG. 1 is a perspective view of two mirror image joint protection devices according to a preferred embodiment of the present invention, shown in use on a user;

FIG. 2 is a front perspective view of one of the joint protection devices in FIG. 1;

FIG. 3 is a rear perspective view of the joint protection device of FIG. 2;

FIG. 4 is a side view of the joint protection device of FIG. 2 with the limb of the user in an extended position;

FIG. 5 is a side view of the joint protection device of FIG. 2 with the limb of the user in a flexed position;

FIG. 6 is a perspective view of an alternative exemplary embodiment of a joint protection device according to the present invention, shown in use on a user; and

FIG. 7 is a perspective view of yet another alternative exemplary embodiment of a joint protection device according to the present invention, shown in use on a user.

### DETAILED DESCRIPTION OF THE PREFERRED EMBODIMENTS

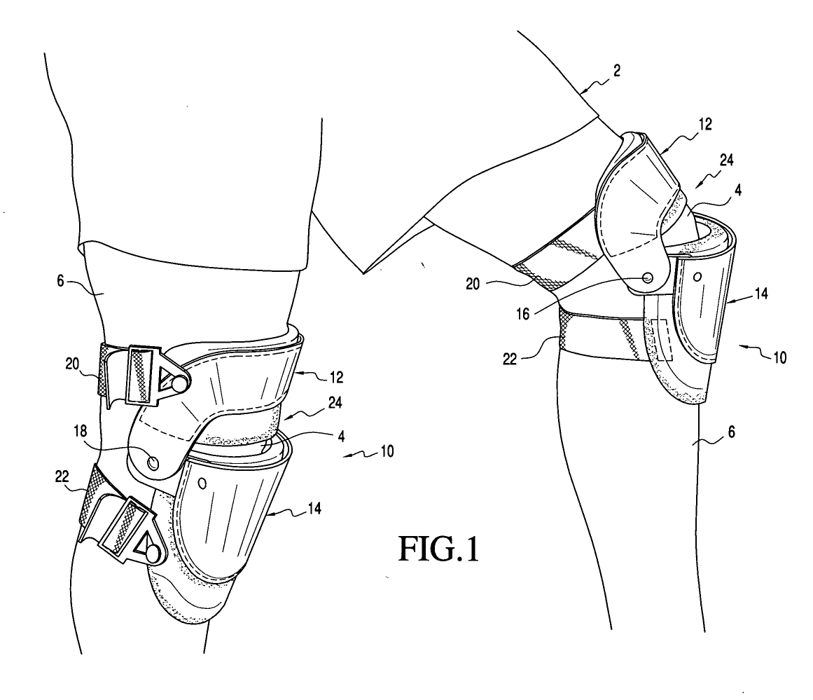

FIG. 1 shows two mirror image joint protection devices 10 according to a preferred embodiment of this invention. The joint protection device 10 is shown as being worn by a user 2 on both knee joints 4. In this exemplary embodiment, the joint protection device 10 is a knee protection device. It will be appreciated that joint protection devices made in accordance with this invention may be applicable to other joints, such as for example the elbow.

The joint protection device 10 includes an upper member 12 and a lower member 14. The upper member 12 is pivotally attached to the lower member 14 at first and second pivot connections 16 and 18. The joint protection device 10 is held adjacent to the limb 6 of the user 2 via first and second attachment members 20 and 22.

The upper and lower members 12 and 14 pivot relative to each other at the pivot connections 16 and 18. The members 12 and 14 pivot with the upper and lower portions of the limb 6 as the limb 6 is extended and flexed.

An opening 24 in the device 10 is defined between the upper and lower members 12 and 14. Advantageously, the opening 24 is operably configured so as to expose the front of the joint 4 when the device 10 is worn. The opening 24 minimizes the overall weight of the device 10. Further, the opening 24 provides for more ventilation of the device 10 to aid in keeping perspiration by the user to a minimum.

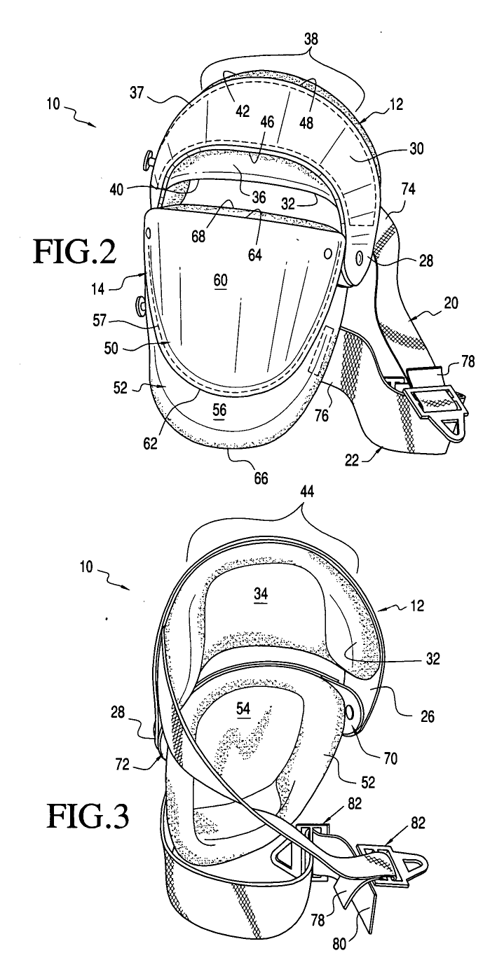

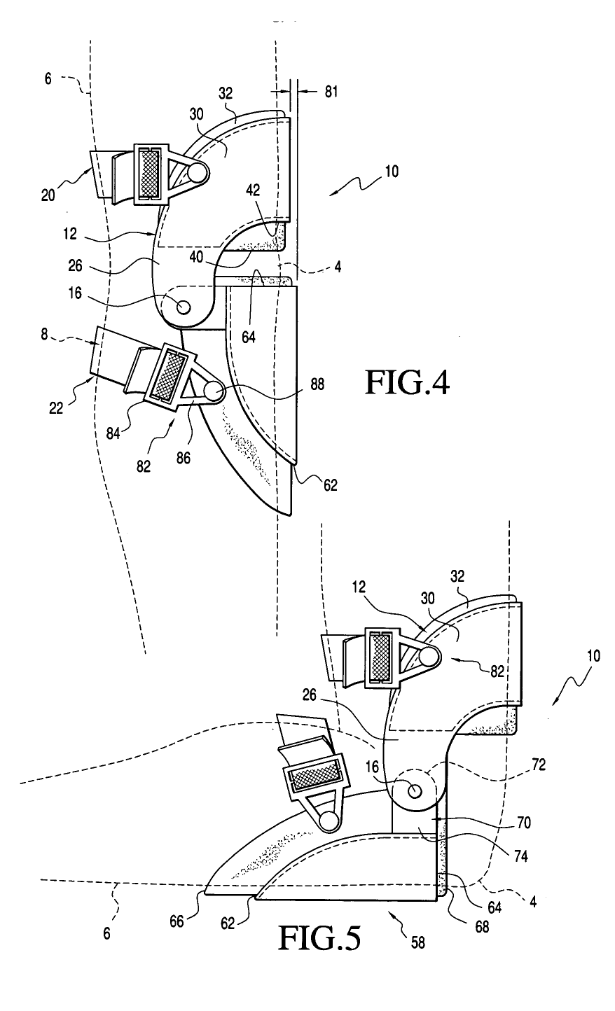

FIGS. 2 and 3 show front and rear prospective views, respectively, of the joint protection device 10. FIGS. 4 and 5 show the same side view of the joint protection device 10, wherein the limb 6 of the user 2 is shown in phantom. FIG. 4 shows the relative positioning of the upper and lower members 12 and 14 when the limb 6 is in an extended position, and FIG. 5 shows the relative positioning when the limb 6 is in a flexed position.

With reference to FIGS. 1-5, the joint protection device 10, which is an exemplary embodiment of a device made in accordance with this invention, will be described below in greater detail.

The upper member 12 has a length defined by first and second end portions or terminals 26 and 28. The first end portion 26 is pivotally connected to the lower member 14 at the first pivot connection 16. The second end portion 28 is pivotally connected to the lower member 14 at the second pivot connection 18. The upper member 12 preferably has a generally inverted u-shape along its length. The general u-shape is defined by the upper member extending from the first end 26 above the joint 4 to the second end 28. When the device 10 is worn, as shown in FIGS. 1, 4 and 5, the upper member 12 is disposable about a portion of the joint 4 such that the upper member 12 covers a portion of the upper section of the limb 6 above the joint 4. The shape of the band 30 will help keep the lower member in a proper placement on the lower portion of the joint.

In this exemplary embodiment, the upper member 12 includes a band 30 and an upper pad 32. The upper pad 32 has an interior surface 34 and an exterior surface 36. The interior surface 34 is operably configured to be adjacent or disposed against the limb 6 when the device 10 is worn. The band 30 is attached to the exterior surface 36. The band 30 is attached, in this exemplary embodiment, by stitching, 37. It should be appreciated that any means of attaching, or combinations thereof, may be employed, including but not limited to gluing, screwing, riveting, hook and loop fasteners, and the like. It should also be appreciated that the upper pad 32 is optional and may be characterized as a support member for band 30.

In this exemplary embodiment, the upper member 12 is preferably made of a material such that the upper member 12 is substantially rigid. Further, it is preferred that the band 30 is made of a substantially rigid material, so as to retain its shape. It is also preferred that the upper pad 32 be made of a cushion-type material, such as but not limited to a conventional foam or gel. It should be appreciated that in other exemplary embodiments the band 30 may not be made rigid and/or solid along its entire length. In other exemplary embodiments, the band includes multiple orifices or slots to increase the ventilation of the device 10.

A central portion 38 of the upper pad 32 has a width defined by a lower edge 40 and an upper edge 42. A central portion 44 of the band 30 has a width defined by a lower edge 46 and an upper edge 48. It is preferred that the lower edge 40 of the central portion 38 of the upper pad 32 is disposed lower, relative to the lower member 14, than the lower edge 46 of the central portion 44 of the band 30. It is preferred that the width of the central portion 38 of the upper pad 32 is greater than the width of the central portion 44 of the band 30. This advantageously provides for a layer of cushion between the band 30 and the skin of the user 2 so as to prevent the device 10 from pinching or “biting” the skin.

The upper member 12 is operably configured so that a lower edge of the upper member 12 rests against the top of the joint 4. In this embodiment, the lower edges 40 and 46 of the upper pad 32 and the band 30 rest against or just above the knee cap of the joint 4 when the device 10 is worn, as shown in FIGS. 1 and 4. This configuration aids in keeping the device 10 from undesirably sliding down the limb 6 of the user 2. Advantageously, the lower edge 40 of the upper pad 32 is disposed between the knee cap and the lower edge 46 of the band 30 when the device is worn, so that the more rigid band 30 does not cause pain to the user 2 by pinching the skin of the user 2.

The lower member 14 is operably configured to cover a lower portion of the joint 4 when the device 10 is worn. See for example, FIGS. 1, 4 and 5. In this exemplary embodiment, the lower member 14 includes an impact member 50. It is preferred that the lower member 14 also include a lower pad 52. The lower pad 52 is operably configured to be disposed between the impact member 50 and the limb 6 when the device is worn. The lower pad 52 has an interior surface 54 and an exterior surface 56. The interior surface 54 is operably configured to be adjacent or disposed against the limb 6 when the device 10 is worn. The impact member 50 is attached to the exterior surface 56. The impact member 50 is attached, in this exemplary embodiment, by stitching, 57. It should be appreciated that any means of attaching, or combinations thereof, may be employed, including but not limited to gluing, screwing, riveting, hook and loop fasteners, and the like. It should also be appreciated that the lower pad is optional.

The impact member 50 is preferably substantially rigid. It is further preferred that the impact member 50 is made of a hard plastic material. It should be appreciated that the invention is not limited to plastic materials.

Preferably, the lower pad 52 is made of a less rigid material than the impact member 50. Further, it is preferred that the lower pad 52 be made of a cushion-type material, such as, but not limited to, a conventional foam or gel. It should be appreciated that in other exemplary embodiments the impact member 50 may not be rigid and/or solid throughout the entire member. Further, in other exemplary embodiments, the impact member 50 includes multiple orifices or slots to further increase the ventilation of the device 10.

The impact member 50 does not significantly extend around the limb 6. Rather, the impact member 50 preferably has a generally arching body with a curved bottom edge so as to conform to the lower front portion of the joint 4, as shown in FIGS. 1-5. It should be appreciated that in other various exemplary embodiments, the impact member may be a variety of different shapes. The impact member 50 is operably configured to cover a front area of the lower portion of the limb below the joint 4. This advantageously enables the device to protect the only portion of the joint that is likely to engage the ground while adding very little weight to the device 10. Preferably, the impact member 50 is operably configured to cover only slightly more than a likely impact area of the joint 4 when the device is worn. The impact area being generally represented by arrow 58, see FIG. 5. The likely impact area 58 is the surface area of the joint that would normally engage the ground or supporting surface, when for example, the user 2 is kneeling. It is preferred that the impact member 50 not cover a significant portion of the limb 6 or joint other than the impact area. Further, the limited size of the impact member 50 enables significant advantages over conventional designs in regards to comfort, weight and ventilation of the overall device 10.

The impact member 50 has an exterior surface 60. The exterior surface 60 is the surface that engages or impacts objects rather than the joint 4. In the present exemplary embodiment, the exterior surface 56 of the lower pad 52 has a surface area that is greater in dimension than an exterior surface 60 of the impact member 50. Having the lower pad 52 being larger than the impact member 50 provides additional protection for the area of the limb that the impact member 50 does not cover. However, it should be understood that having a lower pad that has a larger surface area than the exterior surface of the impact area is optional only, and in other exemplary embodiments, the lower pad may have the same size surface area or perhaps have a slightly smaller surface area. For example, the lower pad 52 in this exemplary embodiment extends further lower and around the leg than the impact member 50. This feature provides additional, but light weight, protection against objects on the ground that may otherwise scrape or press into the skin. This additional protection does not require a rigid member since it is not the main impact area.

The impact member 50 has a lower edge 62 and an upper edge 64. The lower pad has a lower edge 66 and an upper edge 68. A portion of the lower edge 66 of the lower pad 52 extends below the lower edge 62 of the impact member 50 relative to the joint 4. This arrangement advantageously prevents the more rigid impact member 50 from engaging and pinching the skin on the limb 6 of the user 2.

The opening 24 is defined between the upper edge 64 of the impact member and the lower edge 38 of the upper pad 32.

In this exemplary embodiment, the upper edge 64 of the impact member 50 is operably configured to form a horizontal edge, preferably aligned with a midpoint of the joint 4 such that the upper part of the joint 4 is exposed when the device is worn. However, as one of ordinary skill in the art would recognize, the upper edge 64 of impact member 50 may be any shape, such as a gradual arc. A substantial portion of the upper edge 64 of the impact member 50 preferably lies within the same plane, which is perpendicular to a longitudinal axis of the limb 6 when the device is worn. Advantageously, this horizontal edge feature, which enables a significant portion of the joint.4 to be exposed, increases the overall comfort, lightness and ventilation of the device 10.

The lower member 14 further includes first and second arms 70 and 72 as shown in FIG. 3. Each of the arms 70 and 72 include one of the pivot connections 16 and 18, respectively. The arms 70 and 72 each include a free end and a fixed end. The pivot connections 16 and 18 are disposed near the free ends. The arms 70 and 72 are attached to opposing sides of the impact member 50 at the fixed ends. It should be appreciated that in other exemplary embodiments, the arms are formed such that the impact member and the arms are unitary. The arms 70 and 72 are operably configured such that when the device 10 is worn, the pivot connections are substantially aligned with the axis of rotation of the joint 4.

The upper and lower members 12 and 14 are operably configured such that when the device 10 is in an extended position, as shown in FIG. 4, the front edge of the upper member 12 is off-set from the front edge of the lower member as represented by 81. In other words, the lower member 14 extends further from the pivot connections 16 and 18 than the upper member 12. The offset is preferred to be minimal. The offset in this exemplary embodiment may be between about ⅛ to 1½ of an inch.

Advantageously, the offset 81 aides in keeping the device 10 from slipping down the limb 6 when the device 10 is worn. The off-set 81 is operably configured such that, when the device 10 is worn, the lower edge 46 of the band 30 rests on the joint 4, as shown in FIGS. 1 and 4. As discussed above, it is preferred that the lower edge 40 of the upper pad 32 extend below the lower edge 46 such that the padding is disposed between the band 30 and the joint 4 to prevent discomfort.

It is further preferred that the lower pad 52 not extend to the joint 4. In this embodiment, the band 30 of the upper member 12 does not cover any portion of the lower pad. The lower pad 52 and the upper pad 32 may however, in other various exemplary embodiments, be one continuous pad, provided that a significant portion of the joint 4 is exposed. Having the joint 4 exposed advantageously increases the ventilation of the device 10 as well as the overall lightness of the device 10.

The attachment members 20 and 22 are operably configured to releasably attach the device 10 to the limb 6. It should be appreciated that additional attachment members may be utilized in other various exemplary embodiments.

In this exemplary embodiment, each of the attachment members 20 and 22 include a fixed end 74 and 76, respectively, and an opposing attachment end 78 and 80, respectively. The fixed end 74 of attachment member 20 is attached to one side of the upper member 12 and the attachment end 78 is releasably attachable to the other side of the upper member 12. Likewise, the fixed end 76 of attachment member 22 is attached to one side of the lower member 14 and the attachment end 80 is releasably attachable to the other side of the upper member 14.

The upper and lower attachment members 20 and 22 include attachment means 82. The attachment means may be any known or later developed device for adjustably and releasably attaching the attachment ends to the upper and lower members. In this embodiment, each of the attachment means 82 includes a buckle 84 with an engagement triangle 86 and a rod 88. The respective buckles 84 are adjustably attached to the attachment members 20 and 22. The attachment ends 78 and 80 are threaded through the buckles, in a conventional manner, so that the level of tightness of the device 10 can be selectively adjusted. The buckles 84 are conventional buckles that allow a strap to be adjusted to a desired size, wherein when the buckle is in use, the strap will not slip. The rods 88 are fixed to the upper and lower members 12 and 14, respectively. The triangle 86 of each engagement means 82 is operably configured to engage, in a conventional manner, the corresponding rod 88.

It is preferred that the rods 88 and the corresponding fixed end of the attachment members 20 and 22 be disposed on opposing sides of the device 10. It is preferred that the attachment member 20 and 22 not be connected to the device at the very top and bottom edges of the device 10. For example, attachment member 20 is preferably connected to the upper member at a distance, toward the pivot connection 16 and 18, from the upper edge 48 of the central portion 38 of the upper member 12. Similarly, the attachment member 22 is preferably connected to the lower member at a distance, toward the pivot connection 16 and 18, from the lower edge of the lower member 14. The preferable positioning of attachment member 22 is towards the midpoint of lower member 14. However, the position of attachment member 22 can vary somewhat wherein the main objective of the positioning of the attachment member 22 is to keep both upper member 12 and lower member 14 clamped closely to the limb 6 when the joint 4 is straightened. The attachment members are preferred to be connected at a distance from the top and bottom edges of the devices so as to counter the moment imposed on the device by the pivot as the limb moves from the bent to the extended position. This is because as the joint extends, the device will tend to undesirably lift away from the face of the joint. Similarly it is the attachment strap 20 holding the upper member 12 which allows the upper member 12 to advantageously pull up the lower member 14 toward the joint 4 when the limb 6 moves from the extended to a partially or fully bent position.

The particular side of the upper and lower members 12 and 14 to which the fixed ends 74 and 76 of the attachment members 20 and 22 are attached does not matter. The preferred embodiment places the releasably attachable ends to the outside of the joint for easier access and no chance for mutual interference between the releasably attachable ends when the user walks.

Preferably, the upper attachment member 20 is made of a material that has more elasticity than the material of the lower attachment member 22. It is further preferred that the upper attachment member 20 have a low elasticity. The upper attachment member 20 is preferably made of elastic webbing. It is preferred, that when the device 10 is worn, the upper attachment 20 only be tightened a minimal amount to hold the upper member 12 adjacent to the upper section of the limb 6.

Preferably the lower attachment member 22 is made of a material that is substantially non-elastic. This is because the lower section of the limb 6, immediately below the joint 4, has a roughly frustum conical shape. In this embodiment, it is the section just below the knee and above the midpoint of the calf. To aid in keeping the device 10 in a proper height position when worn, the lower attachment member 22, only needs to be tightened to a diameter that is less than the base 8 of the conical shape. In this exemplary embodiment, the lower attachment member 22 is made of nylon webbing or any other suitable material known to those of ordinary skill in the art. However, it should be appreciated that the lower attachment member 22, as well as the upper attachment member 20, may be made of other materials. The lower attachment member 22 may also be made of a rigid adjustable strap, with the appropriate coupling means as are known in the art, such as the rigid straps that are commonly utilized on ski boots and the like.

It should be appreciated that the pivot connections 16 and 18 may be any known or later developed connection that enables relative pivoting motion between the connected members. In this exemplary embodiment, the pivot connections 16 and 18 are blind fasteners that offer the hole a bearing surface. However, as one of ordinary skill in the art would recognize, any type of fastener may be used in the present device, such as a threaded fastener.

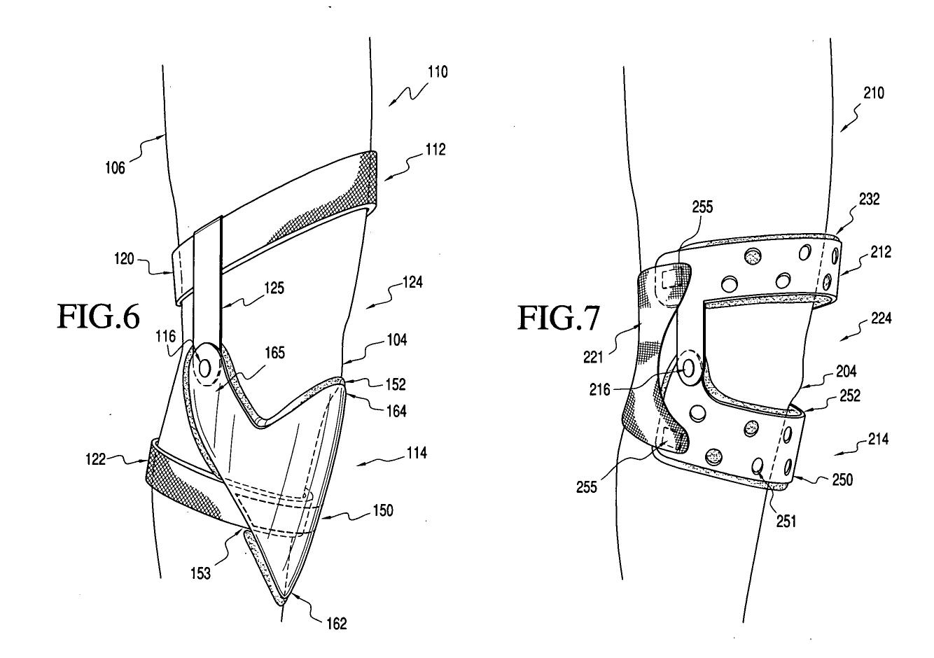

FIG. 6 is a perspective view of an alternative exemplary embodiment of a joint protection device 110 according to the present invention, shown in use on a user. This device 110 is similar to the joint protection device 10 described above in that it includes an upper member 112 and a lower member 114. The device 110 includes the same features and aspects as the device 10, except where noted below and shown in the Figures. The upper member 112 is pivotally attached to the lower member 114 at first and second pivot connections 116 and 118 (not shown). The joint protection device 110 is held adjacent to the limb 106 of the user 102 via first and second attachment members 120 and 122. The opposing side of the device is a mirror image of the side that is visible in FIG. 6.

The upper member 112 and lower members 114 pivot relative to each other at the pivot connection 116 and 118. The members 112 and 114 pivot with the upper and lower portions of the limb 6 as the limb 6 is extended and flexed.

The upper member 112 in the present embodiment may be a support member or strap and may be integrally associated with the attachment means 120. The upper member 114 also includes two arm members 125 designed to be pivotally attached to the lower member 114. Note, only one arm member is visible in FIG. 6, but the other member is on an opposing side of the upper member 112. Upper member 112 in the present exemplary embodiment is made of foam. However, upper member 112 may be made from any material known in the art that will provide enough rigidity to rest atop knee joint 1044 without sliding down limb 106, including, but not limited to, various forms of breathable elastic.

The lower member 114 has an impact member 150. Lower member 114 in this embodiment also includes a lower pad 152.

The impact member 150 has a lower edge 162, an upper edge 164 and an arm 165. In the present preferred alternate embodiment, the lower edge 162 of impact member 150 is V-shaped. However, the lower edge 162 of impact member may be of any shape as long as it protects the user's joint 104 in the impact area as described above. Upper edge 164 has a curved edge relative to the pivot axis of the joint 104. Impact member also has arms 165 that are pivotally connected to the arm members 125 of the upper member 112.

The lower pad 152 has a surface area slightly larger than the impact member 150 for further comfort for the user. Lower pad 152 also may have a channel 153 operably configured to house a portion of the lower attachment member 122. The channel or indentation in other various exemplary embodiments may be spacing between two sections of the lower pads.

Lower attachment member 122 has opposing first and second ends wherein one of the first or second ends is attached to the lower member 114 by any means known to those of ordinary skill in the art including, but not limited to, stitching or hook and loop features. In this embodiment, lower attachment member 122 completely encircles the limb 106. The lower attachment member 122 is operably configured to maintain contact with the skin of the limb, when worn. The lower attachment member 122 maintains contact with the frontal portion of the limb 106 by way of passing through the channel 153 of pad 152. This positioning allows for greater comfort for the user because the attachment means does not leave the attachment to the user's skin and lower pad 152 fits snuggly over the attachment member 122.

It should be appreciated that the lower member, may in other various exemplary embodiments, not include a separate lower pad. In these embodiments, the pad may be integral with the more rigid, protective impact member. With such an arrangement, the channel would be formed within the integral lower member.

It should also be appreciated that the upper attachment member 120, although an integral part of the upper member 112, also completely encircles the limb 106. This is done for the same benefits mentioned above for the lower attachment member 122.

Further, it should be appreciated that the upper and lower attachment members 120 and 122 may be slid on to the limb 106 and then the arm member 125 and the lower member 114 are affixed to the attachment members 120 and 122, respectfully.

FIG. 7 is a perspective view of yet another alternative exemplary embodiment of a joint protection device 210 according to the present invention, shown in use on a user. This device 210 is similar to the joint protection device 10 described above in that it includes an upper member 212 and a lower member 214. The upper member 212 is pivotally attached to the lower member 214 at first and second pivot connections 216 and 218 (not shown). The joint protection device 210 is held adjacent to the limb 206 of the user via a unitary attachment member 221. The opposing side of the device is a mirror image of the side that is visible in FIG. 7.

The upper member 212 and lower member 214 pivot relative to each other at the pivot connection 216 and 218. The members 212 and 214 pivot with the upper and lower portions of the limb 206 as the limb 206 is extended and flexed.

The upper member 212 in the present embodiment is a support member or strap and is be integrally associated with the unitary attachment means 221 and an arm member 226 pivotally attached to the lower member 214. Unitary attachment member 221 is also connected to upper member 212 by any attachment means 255 known in the art. Upper member 212 is made from any material known in the art that would provide enough support to rest atop knee joint 204 without sliding down limb 206. For example, upper member 212 may be made from various forms of breathable elastic.

The upper member also includes a pad 232. Pad 232 provides further comfort for the user.

The lower member 214 has an impact member 250. Lower member 214 also has a lower pad 252.

It should be appreciated that the upper and lower member, may in other various exemplary embodiments, not include a separate lower pad. In these embodiments, the pad may be integral with the more rigid, protective impact member.

Further, it should be appreciated that the upper and lower attachment members 220 and 222 may be slid on to the limb 106 and then the arm member 226 and the lower member 214 are affixed to the attachment members 220 and 222, respectfully.

Impact member has a lower edge 262 and an upper edge 264. In the present preferred alternate embodiment, the lower edge 262 of impact member 150 is preferably substantially horizontal and substantially perpendicular to the user's limb 206. However, the lower edge 262 of impact member may be of any shape as long as it protects the impact area described above. Upper edge 264 is preferably horizontal and substantially perpendicular to the user's limb 106. Impact member is pivotally connected to upper member 212 through arm member 226 of upper member 212.

Lower member 214 has a lower pad 252. Lower pad 214 has a surface area slightly larger than impact member 250. The larger surface area of lower pad 214 provides further comfort for the user. The lower member 214 provides protection for the joint 104 of the user in the impact area. The device 210 is lightweight and does not bite as set forth above.

It should be appreciated that the upper and lower member, may in other various exemplary embodiments, may include ventilation orifices. In these embodiments, the orifices may be integral with the more rigid, protective impact member. With such an arrangement, the orifices would be formed integral with the upper and lower member.

Unitary attachment member 221 is a single piece of elastic or stretchable material that may either integrally attached to upper member 216 and lower member 214 or connected by any attachment means known to those of ordinary skill in the art. The unitary attachment member 221 of device 210 allows for unitary force against the back of joint 204 and therefore allows for greater comfort to the user.

The foregoing description of a preferred embodiment of the invention has been presented for purposes of illustration and description. It is not intended to be exhaustive or to limit the invention to the precise form disclosed. Obvious modifications, variations or combination of embodiments are possible in light of the above teachings. The preferred embodiments were chosen and described to provide an illustration of the principles of the invention and its practical application to thereby enable one of ordinary skill in the art to utilize the invention in various embodiments and with various modifications as are suited to the particular use contemplated. Various changes may be made without departing from the spirit and scope of this invention.

1. A protection device for a joint which pivotally connects an upper and lower section of a limb, the device comprising:
an impact member being substantially rigid and operably configured to cover a lower portion of the joint when the device is worn;
a support member pivotally attached to the impact member by at least two pivot connections, wherein the support member is operably configured to cover a portion of the upper section of the limb above the joint defining an opening in the device between the impact member and the support member such that a front portion of the joint is exposed when the device is worn; and
at least one retaining member, wherein the at least one member is removably connected to at least one of the impact member and support member, the at least one retaining member being operably configured to retain the device adjacent to the joint when the device is worn.

2. The protection device, as recited in claim 1, further comprising a pad disposed on an inner surface of the impact member wherein the pad has a surface area slightly larger than the impact member

3. The protection device, as recited in claim 1, wherein the at least one retaining member further comprise first and second retaining members, the first retaining member being releasably attached to the lower member and the second retaining member being releasably attached to the upper member, wherein when the device is worn the retaining members are operably configured to retain the lower and upper members adjacent to the limb.

4. The protection device for a joint of claim 1 wherein the impact member includes ventilation orifices.

5. A joint protection device for a joint of a limb, comprising:
a lower member being substantially rigid and having two pivot connections, wherein when the device is worn the lower member is operably configured to cover a portion of the joint and the two pivot connections are positioned adjacent opposing sides of the joint;
an upper member having a length defined by opposing ends, the upper member has a generally inverted u-shape disposable about a portion of the joint, and one of the opposing ends is pivotally connected to one of the pivot connections and the other opposing end is pivotally connected to the other pivot connection, wherein when the device is worn the upper member extends from one of the pivot connections above the joint to the other of the pivot connections; and
at least one retaining member being releasably attached to the lower member and the retaining member being releasably attached to the upper member, wherein when the device is worn the retaining member is operably configured to retain the retaining member adjacent to the limb.

6. The protection device, as recited in claim 5, further comprising a pad disposed between the lower member and the limb wherein the pad has a surface area slightly larger than the impact member.

7. The protection device, as recited in claim 5, wherein the at least one retaining member further comprises first and second retaining members, the first retaining member being releasably attached to the lower member and the second retaining member being releasably attached to the upper member, wherein when the device is worn the retaining members are operably configured to retain the lower and upper members adjacent to the limb.

8. The protection device, as recited in claim 5, wherein the impact member includes ventilation orifices

9. A protection device for protecting a joint, which provides a pivotal connection between upper and lower portions of a limb, the device comprising:
a lower member having a substantially rigid shell and a pad, the shell having a dimensional area that is less than a dimensional area of the pad, the pad having interior and exterior surfaces, the interior surface operably configured to be disposed adjacent the joint and the lower portion of the limb when the device is in use, and the shell disposed on the exterior surface;
an upper member rotatably connected to the lower member, wherein the upper member does not cover any portion of the exterior surface of the pad, and when the device is worn the upper member is operably configured to cover a portion of the upper portion of the limb; and,
at least one retaining strap releasably connected to at least one of the lower and upper member and being operably configured to hold the device against the limb when the device is worn.

10. The protection device, as recited in claim 9, wherein the at least one restraining strap comprises two retaining straps releasably connected to at least one of the lower and upper member and being operably configured to hold the device against the limb when the device is worn.

11. The protection device, as recited in claim 9, wherein the impact member includes orifices for increased ventilation.

12. A protection device for protecting a joint, which provides a pivotal connection between upper and lower portions of a limb, the device comprising:
an impact member being substantially rigid and operably configured to cover a lower portion of the joint when the device is worn;
an upper support band pivotally attached to the impact member by at least two pivot connections, wherein the support band is operably configured to cover a portion of the upper section of the limb above the joint defining an opening in the device between the impact member and the support band such that a front portion of the joint is exposed when the device is worn; and
a lower support band, wherein the lower support band is releasably connected to and is operably configured to completely encircle a user's limb.

13. The protection device, as recited in claim 12, further comprising a pad disposed between the impact member and the limb wherein the pad has a surface area slightly larger than the impact member.

14. The protection device, as recited in claim 12, wherein the impact member includes ventilation orifices.

15. A protection device for a joint which pivotally connects an upper and lower section of a limb, the device comprising:
an impact member being substantially rigid and operably configured to cover a lower portion of the joint when the device is worn;
a support member pivotally attached to the impact member by at least two pivot connections, wherein the support member is operably configured to rest upon the upper section of the limb above the joint defining an opening in the device between the impact member and the support member such that a front portion of the joint is exposed when the device is worn; and
at least one retaining member, wherein the at least one member is removably connected to at least one of the impact member and support member, the at least one retaining member being operably configured to retain the device adjacent to the joint when the device is worn.
